(19) United States
(12) Patent Application Publication
(10) Pub. No.: US 2010/0204734 A1
(43) Pub. Date: Aug. 12, 2010

(54) OSTEOSYNTHESIS SYSTEM FOR CONNECTING AT LEAST TWO VERTEBRAE

(75) Inventor: Christian Renaud, Arthes (FR)

Correspondence Address: YOUNG & THOMPSON, 209 Madison Street, Suite 500, Alexandria, VA, 22314, US

(73) Assignees: Christian RENAUD, Arthes (FR); Jean-Francois LIMITO, Balma (FR); CERIA - CONCEPTION ETUDES REALISATION D'IMPLANTS ET D'ANCILLAIRES, Semblancay (FR)

(21) Appl. No.: 12/596,315

(22) PCT Filed: Apr. 18, 2008
(86) PCT No.: PCT/FR2008/050700

§ 371 Date: Apr. 20, 2010

(30) Foreign Application Priority Data
Apr. 19, 2007 (FR) 0754575

(51) Int. Cl. A61B 17/70

(52) U.S. Cl. 606/264

(57) ABSTRACT

An osteosynthesis system (2) for connecting at least two vertebrae, includes attachment members in each vertebra. The attachment members include a first nail (14) to be impact-inserted into a first vertebra (4), a second nail (16) substantially coplanar and substantially parallel to the first nail (14) and to be impact-inserted into a second vertebra (6), and a bridge (18) rigidly connected (18) to the first and second nails (14, 16).

The present invention relates to an osteosynthesis system for connecting at least two vertebrae, of the type comprising fixation elements in each of the vertebrae.

Systems for the osteosynthesis of vertebrae by the posterior route comprising a plate having two holes through which are inserted two fixation screws which form a fixation assembly and which are each to be engaged in a vertebra are already known. Two adjacent vertebrae are thus immobilized relative to each other in order to enable those two vertebrae to fuse together.

However, such a system is difficult to fit.

An object of the invention is to have a system which is easier to fit.

To that end, the invention relates to an osteosynthesis system of the above-mentioned type, characterized in that the fixation elements comprise:

a first nail which is to be inserted into a first vertebra by impact-insertion;

a second nail which is substantially coplanar and substantially parallel with the first nail and which is to be inserted into a second vertebra by impact-insertion; and

a bridge rigidly connected to the first and second nails.

According to particular embodiments, the osteosynthesis system comprises one or more of the following features, taken in isolation or according to any technically possible combination:

each nail is connected to the bridge by a substantially spherical head, which permits the preservation of normal relative movement between the vertebrae in a sagittal plane while at the same time holding them rigidly in order to avoid any displacement out of that plane;

the head of each of the first and second nails has a shape suitable for the impact-insertion and the unscrewing of the nail;

the head comprises at least one unscrewing notch forming engagement surfaces for the unscrewing of the nail;

the system also comprises a third nail which is located between the first and second nails and which is to be inserted into a third vertebra by impact-insertion, the third nail being connected to the first and second nails by the bridge;

the bridge has a flexibility suitable for permitting a bending-extension movement between the vertebrae which receive the nails connected by the bridge;

the bridge has a curved shape;

the bridge is convex towards the distal end of the nails;

the adjacent nails form with each other a non-zero angle, preferably approximately equal to 6°;

each nail is associated with an anchoring dowel, the purpose of the dowel being to expand during the insertion of the nail into the dowel, each nail comprising means for the insertion of the nail into the respective dowel by axial thrust and for the axial retention of the nail in the dowel, the dowel being useful in pedicular fixation in the case of doubtful stability in view of the state of the bone;

the insertion and retention means comprise a thread which has an asymmetrical cross-section and which is in a form such that the nail is capable of being inserted into the dowel by impact-insertion and of being removed from the dowel by unscrewing;

the thread has an inclined distal flank and a substantially radial proximal flank;

the system is produced from a material transparent to X-rays.

The invention will be better understood on reading the following description which is given by way of example and with reference to the appended drawings, in which:

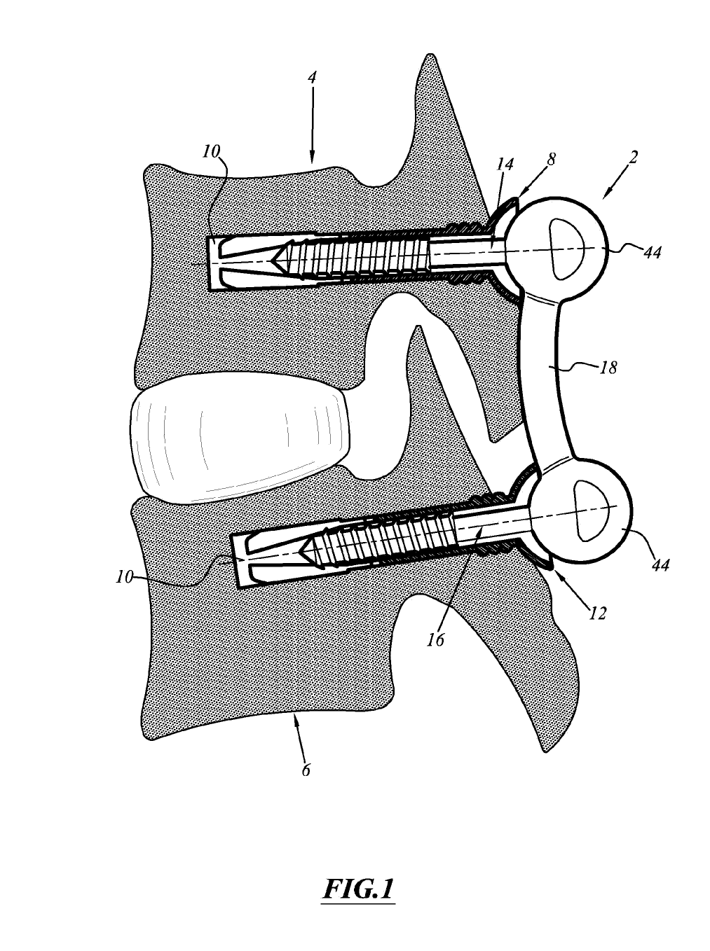

FIG. 1 is a diagrammatic view of a vertical section through an osteosynthesis system according to the invention in place on two vertebrae;

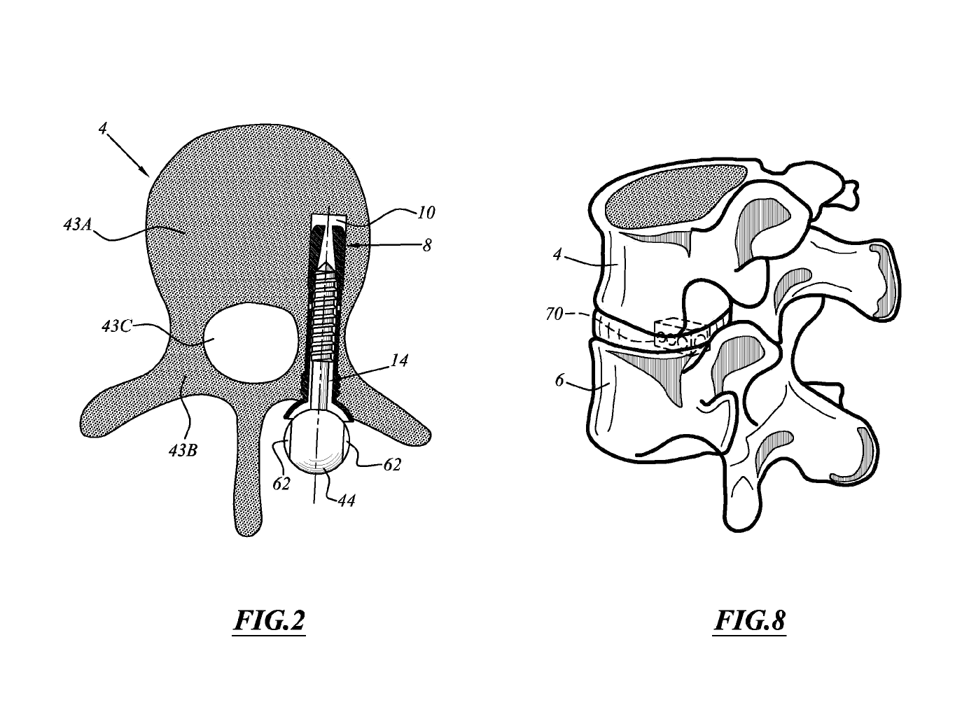

FIG. 2 is a diagrammatic view of a horizontal section through the system of FIG. 1;

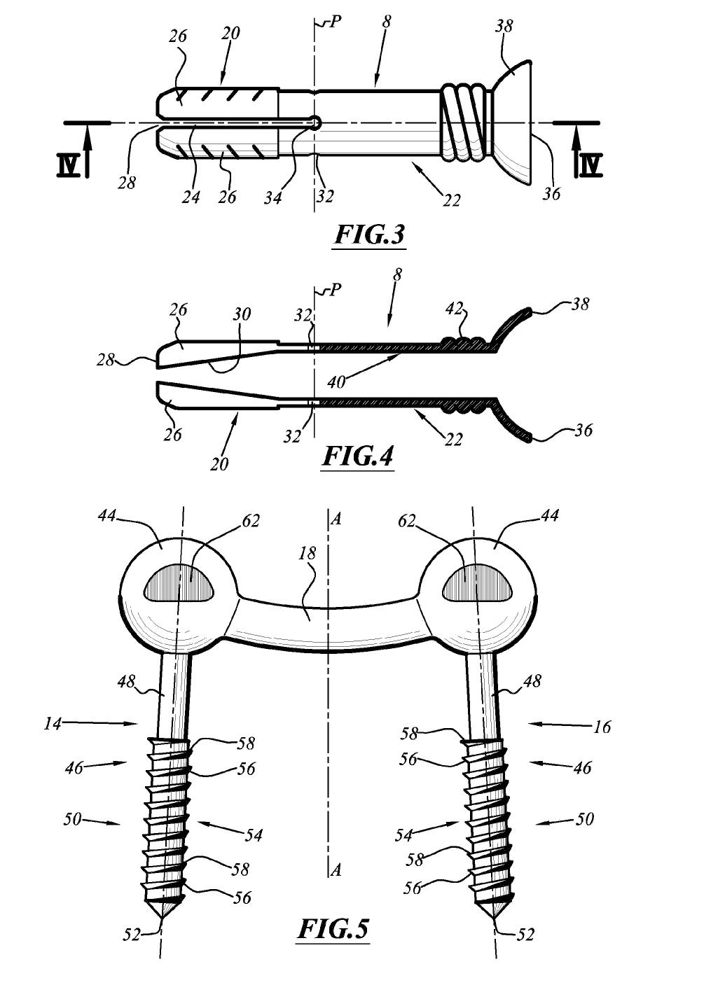

FIG. 3 is a diagrammatic side view of a fixation dowel according to the invention;

FIG. 4 is a diagrammatic view of a section along a centre axis IV-IV of the fixation dowel of FIG. 3;

FIG. 5 is a diagrammatic front view of an assembly of two nails connected by a bridge according to the invention;

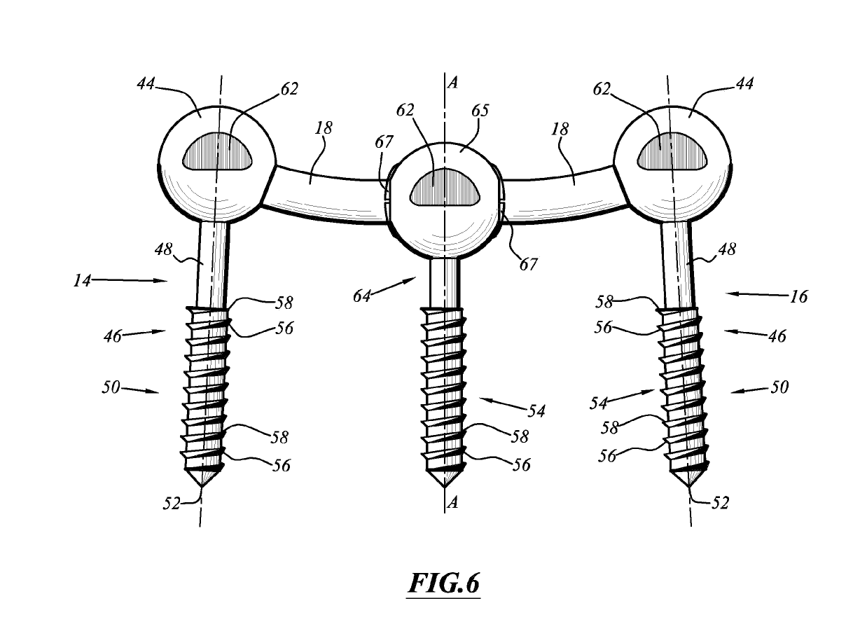

FIG. 6 is an enlarged diagrammatic view similar to FIG. 5 of an assembly of three nails connected by a bridge, according to the invention;

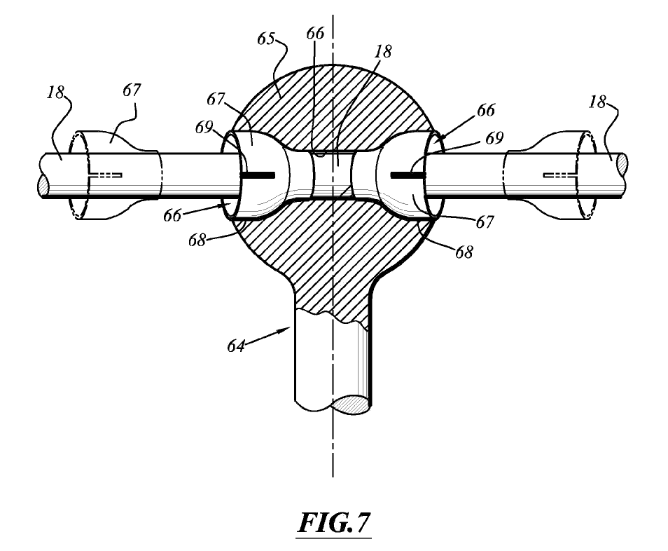

FIG. 7 is an enlarged partially cut-away diagrammatic front view of an intermediate nail of the assembly of FIG. 6; and

FIG. 8 is a diagrammatic perspective view of two vertebrae illustrating the positioning of an intersomatic cage.

FIGS. 1 and 2 illustrate a system for the osteosynthesis by the posterior route of a first 4 and a second 6 vertebra, comprising a first anchoring dowel 8 inserted in a hole 10 formed in the first vertebra 4, a second anchoring dowel 12 inserted in a hole 10 formed in the second vertebra 6, and also a first 14 and a second 16 nail which are to be inserted into the first and second dowels 8, 12, respectively, and which are connected to each other by a flexible bridge 18.

Each nail-dowel fixation assembly 8, 14; 12, 16 forms a fixation assembly in a vertebra 4; 6.

The first and second dowels 8, 12 are identical and are illustrated in more detail in FIGS. 3 and 4.

Throughout the following, “distal” means that which is to be positioned towards the inside of the vertebra and “proximal” that which is to be positioned towards the outside of the vertebra.

Each dowel 8, 12 is formed in one piece from biocompatible plastics material and is made up of an expandable distal part 20 and a proximal part 22.

The distal part 20 comprises an axial slot 24 cutting the distal part 20 into two identical segments 26 and extending as far as a distal end 28 of the dowel 8, 12.

The two segments 26 are intended to spread apart radially from each other when the nail 14, 16 is inserted into the distal part 20 of the dowel 8, 12. For that purpose, the part 20 has an inside diameter decreasing towards the distal end 28 and, in the example illustrated in FIG. 4, forms a frustonconical internal portion 30.

In addition, the two segments 26 each comprise a radial hole 32, the holes 32 being formed radially in the same plane P perpendicular to the axis of the dowel. The holes 32 promote the spreading-apart of the segments 26 in the region of the holes 32 during the insertion of the nail 14, 16.

In the same manner, the slot 24 is terminated on its proximal side by a cylindrical recess 34 forming two holes having the same diameter as the holes 32. The recess 34 is in the same axial plane as the slot 24 and in the same plane P perpendicular to the axis of the dowel 8, 12 as the holes 32.

The proximal part 22 comprises, from the proximal end 36 of the dowel 8, 12 towards the distal part 20, a flared inlet portion 38 which is to guide the nail 14, 16, then a portion 40 which has a constant inside diameter and which connects the flared portion 38 to the distal part 20, as illustrated in FIG. 4.

The portion 40 comprises, at its proximal end, an external thread 42 which projects radially, extending, for example, over approximately 1 centimetre and permitting a pre-anchoring of the dowel 8, 12 in the vertebra 4, 6 before the insertion of the nail 14, 16. In the example illustrated, the thread 42 has a cylindrical profile and a pitch of approximately ⅓ cm. Another type of thread 42 may nevertheless be used.

Over the rest of the portion 40, the external surface is smooth and cylindrical.

As illustrated in FIG. 2, the distal part 20 is to be positioned in the intra-somatic part 43A of the vertebra 4, 6, while the proximal part 22 is to be positioned in the intra-pedicular part 43B of the vertebra 4, 6, the purpose of the part of the portion 40 whose external surface is cylindrical being to extend alongside the intra-pedicular tunnel 43C.

The length of the dowel 8, 12 is, for example, from 30 mm to 40 mm, the proximal part 22 having a length of from 20 to 25 mm, and the distal part 20 having a length of approximately 15 mm.

The outside diameter of the distal part 20 measured at the portion 30 is larger than the outside diameter of the part of the portion 40 whose external surface is cylindrical. The outside diameter of the distal part 20 is, for example, approximately 6 mm, while the part of the portion 40 having a cylindrical external surface has an outside diameter of approximately 5 mm.

FIG. 5 illustrates the first and second nails 14, 16 as well as the bridge 18 connecting them.

The first and second nails 14, 16 are identical. They comprise a head 44 of generally spherical shape and a shank 46 which extends from the head 44.

The head 44 is to be received in the inlet portion 38.

The shank 46 comprises a proximal part 48 whose external surface is cylindrical, and a distal part 50 extending the part 48 to a distal tip 52 of the nail 14, 16.

The distal part 50 comprises a thread 54 of asymmetrical cross-section extending over the entire length of the distal part 50, as far as the tip 52. The thread 54 has a saw-tooth profile and comprises an inclined distal flank 56 and a substantially radial proximal flank 58 which form an angle of from 15 to 60°, preferably from 30 to 45°, with each other. The distal and proximal flanks 56, 58 thus form a triangular saw-tooth profile. Nevertheless, the thread may also have a trapezoidal profile or another appropriate profile.

The inclination of the distal flank 56 relative to the axis of the nail 14, 16 is, for example, from 15 to 75°, preferably from 20 to 40°.

The inclination of the proximal flank 58 relative to the axis of the nail 14, 16 is for its part, for example, from 60 to 90°, preferably from 80 to 88°.

The outside diameter of the distal part 50 is slightly larger than the inside diameter of the proximal part 22 of the dowel 8, 12. The outside diameter of the proximal part 48 is smaller than the outside diameter of the distal part 50 and smaller than the inside diameter of the part 22.

The thread 54 constitutes a means for the insertion of the nail 14, 16 into the dowel 8, 12 by axial thrust, and for axial retention. For, the slight inclination of the distal flank 56 enables the nail 14, 16 to slide over the internal surface of the dowel 8, 12, in the direction oriented towards the distal end 28 of the dowel 8, 12. The great inclination of the proximal flank 58 of the thread 54 prevents the nail 14, 16 from sliding over the internal surface of the dowel, in the direction oriented towards the proximal end 36 of the dowel 8, 12. Thus, the nail 14, 16 is capable of being inserted into the vertebra 4, 6 by impact-insertion or axial translation without rotation. After impact-insertion, the nail 14, 16 is immobilized by the dowel 8, 12 in terms of translation owing to the pressure exerted by the dowel 8, 12 on the distal and proximal flanks 56, 58. In order to remove the nail 14, 16, first of all the bridge 18 is sawn at the head 44, then the nail 14, 16 is unscrewed.

The bridge 18 connects the heads 44 of the first and second nails 14, 16. It is formed by a rod in the shape of an arc of a circle whose radius of curvature is approximately 430 mm and whose axis is perpendicular to the plane containing the axes of the two nails 14, 16. The centre of that arc is located on the proximal side, so that the bridge 18 is convex towards the distal side.

The assembly formed by the bridge 18 and the two nails 14, 16 is, as illustrated in FIG. 5, symmetrical relative to a centre plane A-A located half-way between the two nails 14, 16 and perpendicular to the bridge 18.

The bridge 18 has a circular cross-section of approximately 3 mm. In addition, the bridge is connected to the two heads 44 so that the axes of the nails 14, 16 form with each other an angle of approximately 6°, corresponding to a physiological lumbar angulation between two vertebrae 4, 6.

The bridge 18 is flexible around its initial resting position, shown in FIG. 5, substantially in the plane containing the axes of the two nails 14, 16, owing to its curved shape. On the other hand, the bridge 18 avoids any horizontal translation (parallel with the centre plane A-A) and axial rotation of the nails 14, 16. The head 44 of each nail 14, 16 comprises two notches 62 which are parallel with the axis of the nail and opposite each other and which enable the head 44 to be clamped in order to cause the nail 14, 16 to rotate and thus to unscrew it.

The spherical shape of the head 44 ensures optimum distribution of the mechanical stresses between the bridge 18 and the two nails 14, 16, while at the same time preserving a large capacity for resilient deformation at the junction between the bridge 18 and the head 44.

The flexibility of the bridge 18 is such that the force necessary to move the distal ends of the nails 14, 16 apart or towards each other, starting from their initial resting position in which the axes of the nails 14, 16 form an angle of approximately 6° with each other, as far as a position in which the axes of the nails 14, 16 are parallel, is, for example, from 25 to 50 N.

The two nails 14, 16 and the bridge 18 are, for example, integral with each other and are produced from a material transparent to X-rays, such as Cerlac™.

The osteosynthesis system 2 does not necessarily comprise a dowel 8, 12. The nail-bridge assembly 14, 16, 18 is then, for example, fixed to the vertebrae 4, 6 by means of a suitable cement of known type.

In another embodiment, illustrated in FIGS. 6 and 7, the osteosynthesis system 2 is substantially analogous to the system 2 of the first embodiment but differs basically in that the fixation assembly comprises three nails 14, 16, 64 of similar length, and a single bridge 18 connecting the three nails 14, 16, 64 to each other. The nails 14, 16, 64 comprise an intermediate nail 64 and two end nails 14, 16.

The end nails 14, 16 are substantially identical to the nails 14, 16 of the first embodiment and form an angle of approximately 6° with each other. Their head 44 comprises a hole (not shown) for receiving the bridge 18 in a force-fit.

The bridge 18 is thus fixedly joined to the nails 14, 16. The bridge 18 is similar to the bridge 18 of the previous embodiment but is of a length such that the three nails 14, 16, 64 face three adjacent vertebrae.

The nail 64 is in a form substantially identical to the nails 14, 16 but differs basically by a spherical head 65 which comprises a hole 66 which extends therethrough (see FIG. 7).

The hole 66 receives the bridge 18 in such a manner that the bridge 18 extends freely through the head 65 on both sides.

The system 2 also comprises two fixation collars 67 which are mounted slidingly on the bridge 18, one on each side of the nail 64, and which are suitable for immobilizing the nail 64 in terms of sliding relative to the bridge 18.

FIG. 7 illustrates by a dot-dash line the system before the nail 64 is fixed to the bridge 18. The collars 67 are then spaced from the nail 64.

The hole 66 comprises two flared surfaces 68 for receiving the collars 67 by force-fitting, as illustrated in FIG. 7. For, the surfaces 68 have a shape complementing the collars 67 but have a smaller diameter than the collars 67 so that the fitting of the collars 67 in the hole 66 is effected by force. For that purpose, the collars 67 comprise an axial slot 69 enabling them to be deformed.

The fitting of the collars 67 in the hole 66 is effected by impact-insertion. Once impact-inserted in the hole 66, as illustrated in FIGS. 6 and 7, the nail 64 is fixed to the bridge 18.

The mobility of the nail 64 relative to the bridge 18 facilitates the fitting of the system 2 on three vertebrae 4, 6. The fixing of the nail 64 relative to the bridge 18 is, for example, effected once the insertion of the nails 14, 16, 64 into the vertebrae 4, 6 has been carried out.

This system having three nails therefore enables three vertebrae 4, 6 to be immobilized, this being particularly advantageous, for example, in the case of arthrodesis L5S1 with an intersomatic cage when the inter-superjacent level L4L5 has an impaired distal level.

In another embodiment, the osteosynthesis system 2 having three nails 14, 16, 64 is produced in one piece.

In another embodiment, illustrated in FIG. 8, the osteosynthesis system 2 also comprises an intersomatic cage 70 of known type. The latter is to be inserted between vertebral plates of the first and second vertebrae 4, 6, in order to neutralize intervertebral rotation and thus to ensure immediate stability. Furthermore, the cage 70 provides a “spacer” effect which re-establishes vertebral distraction.

The intersomatic cage 70 is preferably provided with a bone graft, in known manner.

The flexibility of the bridge 18 is selected in such a manner that the osteosynthesis system 2 according to the invention permits a slight bending-extension movement of the two vertebrae 4, 6 relative to each other. In addition, the bridge substantially neutralizes any horizontal translation and axial rotation of the vertebrae relative to each other.

When the system comprises one or more cages 70, the nail-bridge assembly 14, 16, 18 maintains some stress on the bone structures and on the bone graft contained in the cage(s) 70, owing to the flexibility of the bridge 18, with the corollary effect of accelerating the process of osteogenesis and therefore of fusion of the vertebrae 4, 6.

The dowel 8, 12 enables the stresses transmitted by the nail 14, 16 to the vertebra 4, 6 to be absorbed, while at the same time increasing the anchoring of the system 2 at the intra-somatic part 43A of the vertebra 4, 6 since the dowel 8, 12 is provided in order to expand in the intra-somatic part 43A of the vertebra 4, 6 which represents the most efficient and the widest anchoring region in the vertebra 4, 6.

The dowel 8, 12 is also of value in the context of reconstruction after the accidental disintegration of an osteosynthesis system 2 which has brought about a widening of the hole 10. The dowel 8, 12 then enables the nail 14, 16 to be anchored despite the widening of the hole 10.

Finally, the production of the nail-bridge assembly 14, 16, 18 from biocomposite material transparent to X-rays, in particular enables the vertebrae to be seen more clearly.

The dowel 8, 12 can be used in association with a conventional metal osteosynthesis assembly when the quality of the vertebral bone compromises the stability of the assembly.

The dowel 8, 12 is then used as a reinforcement for the metal osteosynthesis.

1. Osteosynthesis system (2) for connecting at least two vertebrae, of the type comprising fixation elements in each of the vertebrae, the fixation elements comprising:
a first nail (14) which is to be inserted into a first vertebra (4) by impact-insertion;
a second nail (16) which is substantially coplanar and substantially parallel with the first nail (14) and which is to be inserted into a second vertebra (6) by impact-insertion; and
a bridge (18) rigidly connected to the first and second nails (14, 16),
characterized in that each nail (14, 16) is connected to the bridge (18) by a substantially spherical head (44).

2. Osteosynthesis system according to claim 1, characterized in that the head (44, 45) of each of the first and second nails (14, 16) has a shape suitable for the impact-insertion and the unscrewing of the nail (14, 16).

3. Osteosynthesis system according to claim 2, characterized in that the head (44, 65) comprises at least one unscrewing notch (62) forming engagement surfaces for the unscrewing of the nail (14, 16).

4. Osteosynthesis system (2) according to claim 1, characterized in that it also comprises a third nail (64) which is located between the first and second nails (14, 16) and which is to be inserted into a third vertebra by impact-insertion, the third nail (64) being connected to the first and second nails (14, 16) by the bridge (18).

5. Osteosynthesis system (2) according to claim 1, characterized in that the bridge (18) has a flexibility suitable for permitting a bending-extension movement between the vertebrae (4, 6) which receive the nails (14, 16, 64) connected by the bridge (18).

6. Osteosynthesis system (2) according to claim 1, characterized in that the bridge (18) has a curved shape.

7. Osteosynthesis system (2) according to claim 6, characterized in that the bridge (18) is convex towards the distal end of the nails (14, 16, 64).

8. Osteosynthesis system (2) according to claim 1, characterized in that the adjacent nails (14, 16, 64) form with each other a non-zero angle, preferably approximately equal to 6°.

9. Osteosynthesis system (2) according to claim 1, characterized in that each nail (14, 16, 64) is associated with an anchoring dowel (8, 12), the purpose of the dowel (8, 12) being to expand during the insertion of the nail (14, 16, 64) into the dowel (8, 12), each nail (14, 16, 64) comprising means for the insertion of the nail (14, 16, 64) into the respective dowel (8, 12) by axial thrust and for the axial retention of the nail (14, 16, 64) in the dowel (8, 12).

10. Osteosynthesis system (2) according to claim 9, characterized in that the insertion and retention means comprise a thread (54) which has an asymmetrical cross-section and which is in a form such that the nail (14, 16, 64) is capable of being inserted into the dowel (8, 12) by impact-insertion and of being removed from the dowel (8, 12) by unscrewing.

11. Osteosynthesis system (2) according to claim 10, characterized in that the thread (54) has an inclined distal flank (56) and a substantially radial proximal flank (58).

12. Osteosynthesis system according to claim 1, characterized in that it is produced from a material transparent to X-rays.
